(19) United States
(12) Patent Application Publication
(10) Pub. No.: US 2010/0236975 A1
(43) Pub. Date: Sep. 23, 2010

(54) METHODS FOR PREDICTING OXALATE AND STRUVITE STONE FORMATION IN ANIMALS

(75) Inventor: Ryan Michael Yamka, Topeka, KS (US)

Correspondence Address: COLGATE-PALMOLIVE COMPANY, 909 RIVER ROAD, PISCATAWAY, NJ, 08855, US

(73) Assignee: HILL'S PET NUTRITION, INC., Topeka, KS (US)

(21) Appl. No.: 12/294,529

(22) PCT Filed: Mar. 27, 2007
(86) PCT No.: PCT/US2007/007499

§ 371 Date: Sep. 25, 2008

Related U.S. Application Data
(60) Provisional application No. 60/786,418, filed on Mar. 27, 2006.

(51) Int. Cl. B65D 69/00; G06F 19/00; G01N 33/48

(52) U.S. Cl. 206/569; 702/19

(57) ABSTRACT

The invention provides methods for predicting oxalate and struvite stone formation in an animal by determining the amount of selected nutrients in a food for consumption by the animal, the amount of selected urine metabolites, and/or the urine pH for the animal and predicting oxalate and/or struvite stone formation using a formula that equates the amount of such nutrients, metabolites, or urine pH to the likelihood that an animal will form oxalate and/or struvite stones.

### CROSS-REFERENCE TO RELATED APPLICATIONS

The present application claims priority to and the benefit of U.S. Provisional Application Ser. No. 60/786,418 filed Mar. 27, 2006 and International Patent Application PCT/US2007/007499 filed Mar. 27, 2007, the entirety of which is hereby incorporated by reference.

### BACKGROUND OF THE INVENTION

This invention relates generally to methods for predicting oxalate and struvite stone formation in animals and particularly to methods for predicting oxalate and struvite stone formation in an animal based upon the composition of a food consumed by an animal, the metabolites in an animal's urine, the animal's urine pH, or a combination thereof.

Urolithiasis is the presence of stones and the process of forming stones in the urinary tract, i.e., the kidney, bladder, and/or urethra. Struvite uroliths are stones in the urinary tract comprising the mineral struvite or magnesium ammonium phosphate hexahydrate. Calcium oxalate uroliths are stones in the urinary tract composed of the mineral calcium oxalate. These uroliths or stones are also referred to as calculi.

The formation of oxalate and struvite stones in the urinary tract is a significant clinical problem for animals, including companion animals such as dogs and cats. Bacterial urinary tract infection is an important predisposing factor for struvite uroliths. The cause of calcium oxalate uroliths remains unknown. Animal urine pH has been shown to be an important determinant in the prevention and treatment of stone formation. A reduction in urine pH has been shown to reduce the incidence of struvite uroliths. However, a decrease in urine pH may increase the risk of calcium oxalate uroliths.

Methods for predicting the formation of stones in animals are known. A commonly used program to predict stone formation by measuring urinary super saturation, EQUIL2 (Werness et al., J Urol. 1985 December; 134(6):1242-4), is available to skilled artisans. This program determines the relative super saturation (RSS) of various salts and acids. RSS values >1 defines urine that is supersaturated with the salt in question, i.e., the potential to form a particular stone. There are, however, problems with the program. To predict RSS, this program requires the measurement of numerous factors, i.e., urine pH and the concentrations of calcium, magnesium, sodium, potassium, ammonium, phosphate, oxalate, citrate, sulfate, uric acid and chloride (Robertson. W. G., J. S. Jones, M. A. Heaton. A. E. Stevenson and P. J. Markwell. 2002. Predicting the crystallization of urine from cats and dogs with respect to calcium oxalate and magnesium ammonium phosphate (struvite). J. Nutr. 132:1637 S-1641S.). The requirement that all these metabolites be measured requires a lot of time and is expensive. Finding a way to predict oxalate and struvite stone formation using the nutrient content of foods, the metabolite content of urine, and urine pH, particularly if only a few parameters had to be measured, would simplify the process, reduce the number of animals required for studies for developing and evaluating animal foods, and reduce the costs associated with diagnosing and treating diseases and conditions associated with the formation of oxalate and struvite stones. There is, therefore, a need for new methods for predicting stone (oxalate and struvite) formation.

### SUMMARY OF THE INVENTION

It is, therefore, an object of the present invention to provide methods and devices for predicting oxalate and struvite stone formation in an animal.

It is another object of the invention to provide articles of manufacture in the form of devices and kits that are useful for predicting oxalate and/or struvite stone formation.

These and other objects are achieved using novel methods for predicting oxalate and struvite stone formation in an animal by determining the amount of selected nutrients in foods, the amount of selected metabolites in urine, and/or urine pH and predicting the likelihood of oxalate stone formation using a formula that equates the amount of all or a subset of these parameters to the likelihood of oxalate stone formation. Devices useful for predicting oxalate and/or struvite stone formation, kits comprising devices useful for predicting oxalate and/or struvite stone formation, and various means for communicating information about or instructions for using the present invention are also provided.

Other and further objects, features, and advantages of the present invention will be readily apparent to those skilled in the art.

### DETAILED DESCRIPTION OF THE INVENTION

The term “animal” means a human or other animal susceptible to the formation of oxalate and/or struvite stones, including avian, bovine, canine, equine, feline, hicrine, murine, ovine, and porcine animals. Preferably, the animal is a canine or feline, most preferably a feline such as a cat.

The term “companion animal” means an individual animal of any species kept by a human caregiver as a pet, or any individual animal of a variety of species that have been widely domesticated as pets, including dogs (Canis familiaris) and cats (Felis domesticus), whether or not the individual animal is kept solely or partly for companionship. Thus “companion animals” herein include working dogs, farm cats kept for rodent control, etc., as well as pet dogs and cats.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof.

The term “virtual package” means that the components of a kit are associated by directions on one or more physical or virtual kit components instructing the user how to obtain the other components. e.g., in a bag containing one component and directions instructing the user to go to a website, contact a recorded message, view a visual message, or contact a caregiver or instructor to obtain instructions on how to use the kit.

The symbol “*” in the formulas herein means that the elements on each side of the “*” are multiplied, e.g., (1.2*potassium) means that the amount of potassium in the food (e.g., molar or percentage on dry matter basis) is multiplied by 1.2.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a method” or “a food composition” includes a plurality of such methods or compositions. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

In one aspect, the present invention provides a method for predicting oxalate stone formation in an animal. The method comprises determining the amount of selected nutrients in a food for consumption by the animal and predicting oxalate stone formation using a formula that equates the amount of such nutrients to the likelihood of oxalate stone formation in an animal.

In one embodiment, the invention provides a method for predicting oxalate stone formation in an animal comprising determining the amount of calcium, chloride, and potassium in a food for consumption by the animal and predicting oxalate stone formation using the formula (Formula 1):

LF=FA−(FB*potassium)+(FC*chloride)−(FD*calcium), where FA is from about

14 to about 26; FB is from about 9 to about 29; FC is from about 1 to about 5; and

FD is from about 1 to about 5.

If LF is greater than or equal to 5, the animal is likely to form oxalate stones. Based upon this prediction, the animal can be prescribed preventive therapy program useful for preventing the formation of oxalate stones, e.g., drugs known by those of skill in the art useful for preventing stone formation or a food or diet containing nutrients useful for preventing stone formation.

In another aspect, the present invention provides a method for predicting oxalate stone formation in an animal. The method comprises determining the amount of selected metabolites in a urine sample from the animal and predicting oxalate stone formation using a formula that equates the amount of such metabolites to the likelihood of oxalate stone formation in an animal.

In one embodiment, the invention provides a method for predicting oxalate stone formation in an animal comprising determining the amount of magnesium, oxalate, and citrate in an animal's urine and predicting oxalate stone formation using the formula (Formula 2):

LU=UA+(UB*magnesium)+(UC*oxalate)−(UD*citrate), where UA is from

about 0.6 to about 1.0; UB is from about 200 to about 600; UC is from about 2000 to

about 3600; and UD is from about 300 to about 600.

If LU is greater than or equal to 5, the animal is likely to form oxalate stones. Based upon this prediction, the animal can be prescribed preventive therapy program useful for preventing the formation of oxalate stones, e.g., drugs useful for preventing stone formation or a food or diet containing nutrients useful for manipulating urine metabolite composition and therefore for preventing the formation of oxalate stones.

In another aspect, the present invention provides a method for predicting oxalate stone formation in an animal. The method comprises determining the amount of selected metabolites in a urine sample from the animal, determining the amount of selected nutrients in a food for consumption by the animal, and predicting oxalate stone formation using a formula that equates the amount of such nutrients and metabolites to the likelihood of oxalate stone formation in an animal

In one embodiment, the invention provides a method for predicting oxalate stone formation in an animal comprising determining the amount of magnesium, oxalate, and citrate in an animal's urine, determining the amount of potassium in a food for consumption by the animal, and predicting oxalate stone formation using the formula (Formula 3):

LUF=UF+(UFA*magnesium)+(UFB*oxalate)−UFC*citrate)−

(UFD*potassium), where UF is from about 2 to about 9; UFA is from about 100 to

about 200; UFB is from about 1200 to about 3600; UFC is from about 200 to about

700; and UTD is from about 2 to about 6.

If LUF is greater than or equal to 5, the animal is likely to form oxalate stones. Based upon this prediction, the animal can be prescribed preventive therapy program useful for preventing the formation of oxalate stones, e.g., drugs useful for preventing stone formation or a food or diet containing increased or decreased amounts of potassium and other ingredients useful for altering the urine metabolite composition and therefore for preventing the formation of oxalate stones.

In another aspect, the present invention provides a method for predicting struvite stone formation in an animal. The method comprises determining the amount of selected metabolites in a urine sample from the animal, determining the urine pH for the animal, and predicting struvite stone formation using a formula that equates the amount of such metabolites and urine pH to the likelihood of struvite stone formation in an animal.

In one embodiment, the invention provides a method for predicting struvite stone formation in an animal comprising determining the amount of chloride, magnesium, and citrate in an animal's urine, determining the animal's urine pH, and predicting oxalate stone formation using the formula (Formula 4):

LUU=UU+(UUA*Urine pH)+(UUB*chloride)−UUC*magnesium)−

(UUD*citrate), where UU is from about −140 to about −180; UUA is from about 20

to about 30; UUB is from about 10 to about 30; UUC is from about 1100 to about

2900; and UUD is from about 1000 to about 2000.

If LUU is greater than or equal to 10, the animal is likely to form struvite stones. Based upon this prediction, the animal can be prescribed preventive therapy program useful for preventing the formation of struvite stones, e.g. drugs known by those of skill in the art useful for preventing stone formation or a food or diet containing ingredients useful manipulating for urine pH and urine metabolite composition and therefore for preventing the formation of struvite stones.

In one aspect, the present invention provides a method for predicting oxalate stone formation in an animal comprising determining the amount of selected nutrients in a food for consumption by the animal, determining the amount of selected metabolites in a urine sample from the animal, and predicting oxalate stone formation using two or more formulas that equates the amount of such nutrients and metabolites to the likelihood of oxalate stone formation in an animal, e.g. Formulas 1 and 2, Formulas 1 and 3, Formulas 2 and 3, or Formulas 1, 2, and 3.

In another aspect, the present invention provides a method for predicting oxalate and struvite stone formation in an animal. The method comprises determining the amount of selected metabolites in a urine sample from the animal determining the urine pH for the animal, and predicting oxalate and struvite stone formation using at least two formulas that equates the amount of such metabolites and urine pH to the likelihood of oxalate and struvite stone formation in an animal. e.g. Formulas 2 and 4.

In still another aspect, the present invention provides a method for predicting oxalate and struvite stone formation in an animal. The method comprises determining the amount of selected nutrients in a food for consumption by the animal, determining the amount of selected metabolites in a urine sample from the animal determining the urine pH for the animal, and predicting oxalate and struvite stone formation using at two or more formulas that equates the amount of such nutrients, metabolites and urine pH to the likelihood of oxalate and struvite stone formation in an animal, e.g., Formulas 1 and 4, Formulas 3 and 4, Formulas 1, 3 and 4, or Formulas 1, 2, 3 and 4.

The invention is based upon the novel discovery that the amount of a limited number of certain nutrients in a food and/or certain metabolites in an animal's urine, in combination with urine pH in some circumstances, can be used to predict the likelihood that an animal will form oxalate and/or struvite stones and the discovery of the formulas or algorithms that equates such amounts to the likelihood that an animal will form oxalate and/or struvite stones. The formulas were determined based upon the statistical analysis of the correlation of various nutrients, metabolites, and urine pH values to oxalate and struvite stone formation in test animals. The invention is useful for simplifying the process for predicting oxalate and struvite stone formation, reducing the number of animals required for studies designed to develop and/or evaluate animal foods useful for avoiding stone formation, and reducing the costs associated with diagnosing and treating diseases and conditions associated with the formation of oxalate and struvite stones.

The methods of the present invention are particularly suitable for use with animals that are under the supervision of an animal caregiver, e.g., companion animals such as dogs and cats. The caregiver can determine the likelihood that an animal will form oxalates or struvite stones and take action to mitigate stone formation using a therapy program, e.g., by controlling amount of and/or the nutrient content of the food consumed by the animal.

Methods for determining nutrient amounts in food and other compositions, metabolite amounts in urine, and urine pH are well known to skilled artisans.

The amounts of nutrients are given herein as a percent of the food composition as measured on a dry matter basis and the concentration of urine metabolites are given herein as molar concentrations, unless specifically stated otherwise.

In another aspect, the present invention provides a device useful for predicting oxalate and struvite stone formation in an animal. The device comprises a means for predicting oxalate and/or struvite stone formation in an animal that uses one or more of Formula 1, Formula, 2, Formula 3, and Formula 4 to predict oxalate and/or struvite stone formation in the animal. The appropriate Formula (s) are made available to an operator or are incorporated or programmed into the device and an operator inputs data relating to nutrients, metabolites, and/or urine pH as appropriate for the Formula(s). The result of the calculation based upon the nutrients, metabolites, and/or urine pH is used to predict the likelihood of oxalate or struvite stone formation. The means can be any suitable means for performing routine calculations such as a prewritten document, website, software program, calculator, or computer that is designed or preprogrammed to predict oxalate and/or struvite stone formation based upon one or more of the Formulas. In certain embodiments, the device accepts input from a user comprising the amount of or value for one or more of, as appropriate for the method and parameter (urine or food): potassium, chloride, and calcium (Formula 1); magnesium, oxalate, and citrate (Formula 2); magnesium, oxalate, citrate, and potassium (Formula 3); and Urine pH, chloride, magnesium, and citrate (Formula 4) and utilizes all or a subset of the input and one or more of Formula 1, Formula 2, Formula 3, and Formula 4 to predict oxalate and/or struvite stone formation. In one embodiment, the device is a software program and/or digital media containing such software program designed to permit a user to input data about the ingredients, metabolites, and urine pH and calculate the predicted stone formation using a formula of the present invention. In other embodiments, the device is a computer, calculator, website, or similar device, particularly one that incorporates or utilizes the software program. In a particularly useful embodiment, the device is a computer containing software that displays a user interface for accepting input from an operator comprising the nutrient, metabolite, or urine pH values. The software accepts user input and uses one or more of Formula 1, Formula 2, Formula 3, and Formula 4, as selected by the operator based upon preference, to calculate the likelihood that an animal will form oxalate and/or struvite stones. The software displays the results in any suitable form, e.g., a screen display or printout. In one embodiment, the computer is coupled to one or more of the analytical instruments capable of determining food nutrient content, urine metabolite concentration, or urine pH. The instrument measures the food nutrient content, urine metabolite concentration, or urine pH, as instructed, and feeds the results directly into the computer containing the formulas of the present invention. The computer then calculates whether the animal is likely to form oxalate or struvite stones and displays the results to the instrument and computer operator.

In a further aspect, the present invention provides a kit useful for predicting oxalate and/or struvite stone formation in an animal comprising in separate containers in a single package or in a virtual package, as appropriate for the kit component, a device of the present invention and one or more of (1) a food suitable for animal consumption, (2) a urine pH diagnostic device, (3) a means for communicating information about or instructions for using urine pH diagnostic devices, and (4) a means for communicating information about or instructions for using the methods, devices, and kits of the present invention to predict oxalate and/or struvite stone formation. The kit components are typically in a separate package, in or on the package with one of the other kit components, or in a virtual package, as appropriate for the type of kit component. When the kit comprises a virtual package, the kit is limited to the instructions in a virtual environment in combination with one or more of the other physical kit components.

In another aspect, the present invention provides a means for communicating information about or instructions for (1) using the methods, devices, and kits of the present invention to predict oxalate and/or struvite stone formation in an animal and (2) using urine pH diagnostic devices to determine urine pH. The communicating means comprises a document, digital storage media, optical storage media, audio presentation, or visual display containing the information or instructions. Preferably, the communication is a displayed website or a brochure, product label, package insert, advertisement, or visual display containing such information or instructions. Useful information and instructions include, but are not limited to, contact information for animals or their caregivers to use if they have a question about the invention and its use and how to use the present invention to predict oxalate and/or struvite formation in an animal. The communication means is useful for instructing an animal or its caregiver on the benefits of using the present invention.

In another aspect, the present invention provides use of the Formulas of the present invention to predict the likelihood that an animal will develop oxalate and/or struvite stones. The present invention also provides for the use of the methods, devices, and kits of the preset invention to predict oxalate and/or struvite stone formation in an animal,

The composition of foods suitable for consumption by an animal is known to skilled artisans. Typical food ingredients include but are not limited to fats, carbohydrates, proteins, fibers, and nutrients such as vitamins, minerals, and trace elements. Skilled artisans can select the amount and type of food ingredients for a typical food based upon the dietary requirements of the animal, e.g., the animal's species, age, size, weight, health, and function.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

RSS studies utilizing 13 dry foods varying in nutrients are fed to cats to determine the effect on RSS for magnesium struvite and calcium oxalate stone formation. Each study is designed to determine urine pH, RSS for struvite, and RSS for oxalate as well as various urine metabolites including citrate, magnesium, chloride, oxalate, sodium, phosphate, ammonia, potassium and calcium. Each study consists of ten cats for a total of 130 cats.

Urine samples are collected once per day during the course of the two week study at the end of 24 hours on days 13, and 14. Total urine samples are collected from each cat into a numbered cup. Urine pH is measured via a pH meter.

All feed samples are analyzed for chloride, magnesium, citrate, oxalate, sodium, phosphate, calcium, potassium, and ammonia using methods known to skilled artisans, e.g., methods according to the Association of Official Analytical Chemists; Horn & Squire in Clin. Chim. Acta 1967, 17, 99: Welshman & McCambridge in Clin. Chim. Acta 1973, 46, 243; Toftegaard in J. Clin. Lab. Invest. 1976, 36, 513; Nicar, Skurla, Sakhaee & Pak in Urology 1983, 21, 8; and Warty. Busch & Virji in Clin. Chem. 1984, 30, 1231.

After the conclusion of the study, the nutrients, metabolites, and urine pH values are used to challenge previously published models. The data is then analyzed and a new model is developed using the values that are useful for predicting oxalate and struvite stone formation. Stepwise regression analysis, techniques known to skilled artisans, are used to determine which nutrients, metabolites, or urine pH are of predictive importance and to determine the formulas, constants and coefficients, including their ranges, useful for predicting oxalate and struvite stone formation. The nutrients, analyzed nutrient values, RSS observed values, and RSS predicted values for the experimental foods and the results of the analysis are shown in Tables 1 through 4. The analysis results in the selection of nutrients, metabolites, and urine pH and the predictive equations of the present invention.

TABLE 1
Nutrients in Foods, Percent on a Dry Matter Basis

| Ingredient | 1 | 2 | 3 | 4 | 5 | 6 |
|---|---|---|---|---|---|---|
| Calcium | 0.801 | 0.801 | 0.835 | 0.131 | 1.058 | 0.686 |
| Phosphorous | 0.691 | 0.691 | 0.707 | 0.677 | 1.058 | 0.697 |
| Chloride | 0.724 | 0.724 | 1.039 | 0.950 | 1.080 | 0.836 |
| Magnesium | 0.063 | 0.063 | 0.061 | 0.076 | 0.073 | 0.086 |
| Potassium | 0.614 | 0.614 | 0.749 | 0.819 | 0.713 | 0.793 |
| Sodium | 0.296 | 0.296 | 0.300 | 0.360 | 0.410 | 0.322 |
| Protein | 34.372 | 34.372 | 33.822 | 34.833 | 38.851 | 35.184 |

| Ingredient | 7 | 8 | 9 | 10 | 11 |
|---|---|---|---|---|---|
| Calcium | 0.868 | 0.925 | 0.858 | 0.846 | 0.855 |
| Phosphorous | 0.727 | 0.807 | 0.719 | 0.738 | 0.747 |
| Chloride | 1.031 | 2.163 | 0.912 | 0.933 | 0.899 |
| Magnesium | 0.061 | 0.071 | 0.062 | 0.082 | 0.082 |
| Potassium | 0.847 | 1.055 | 0.848 | 0.814 | 0.812 |
| Sodium | 0.369 | 0.947 | 0.354 | 0.336 | 0.346 |
| Protein | 35.240 | 36.436 | 33.666 | 34.140 | 34.322 |

| Ingredient | 12 | 13 | Min | Max | Std Dev | Average |
|---|---|---|---|---|---|---|
| Calcium | 0.927 | 0.978 | 0.131 | 1.058 | 0.224 | 0.764 |
| Phosphorous | 0.741 | 0.859 | 0.677 | 1.058 | 0.103 | 0.753 |
| Chloride | 0.992 | 0.913 | 0.724 | 2.163 | 0.362 | 0.994 |
| Magnesium | 0.060 | 0.075 | 0.060 | 0.086 | 0.009 | 0.070 |
| Potassium | 0.839 | 0.804 | 0.614 | 1.055 | 0.112 | 0.781 |
| Sodium | 0.392 | 0.435 | 0.296 | 0.947 | 0.171 | 0.390 |
| Protein | 33.697 | 32.866 | 32.866 | 38.851 | 1.517 | 34.619 |

TABLE 2
Urine Metabolites, Molar (M) and Percent on a Dry Matter Basis (%)

| Metabolite | 1 | 2 | 3 | 4 | 5 | 6 |
|---|---|---|---|---|---|---|
| Chloride M | 0.3340 | 0.3398 | 0.3627 | 0.3264 | 0.3452 | 0.3073 |
| Magnesium M | 0.0053 | 0.0058 | 0.0053 | 0.0054 | 0.0059 | 0.0043 |
| Citrate M | 0.0010 | 0.0012 | 0.0009 | 0.0016 | 0.0011 | 0.0017 |
| Oxalate M | 0.00182 | 0.00186 | 0.00152 | 0.00175 | 0.00136 | 0.00207 |
| Sodium M | 0.1473 | 0.1493 | 0.1485 | 0.1376 | 0.1639 | 0.1491 |
| Phosphate M | 0.0715 | 0.0722 | 0.0572 | 0.0665 | 0.0605 | 0.0655 |
| Calcium M | 0.0012 | 0.00108 | 0.00116 | 0.00102 | 0.00368 | 0.00089 |
| Potassium % | 0.19277 | 0.18316 | 0.21517 | 0.20788 | 0.20469 | 0.22381 |
| Ammonia % | 0.2302 | 0.2314 | 0.2527 | 0.2379 | 0.2396 | 0.2306 |

| Metabolite | 7 | 8 | 9 | 10 | 11 |
|---|---|---|---|---|---|
| Chloride M | 0.3639 | 0.4117 | 0.287 | 0.2683 | 0.2774 |
| Magnesium M | 0.0039 | 0.0031 | 0.0046 | 0.0048 | 0.0044 |
| Citrate M | 0.0008 | 0.0005 | 0.0019 | 0.0018 | 0.0020 |
| Oxalate M | 0.00128 | 0.00098 | 0.00083 | 0.00098 | 0.001 |
| Sodium M | 0.1800 | 0.2604 | 0.1322 | 0.1129 | 0.1274 |
| Phosphate M | 0.0598 | 0.059 | 0.043 | 0.0547 | 0.0556 |
| Calcium M | 0.0011 | 0.0095 | 0.00127 | 0.00131 | 0.00118 |
| Potassium % | 0.19713 | 0.15957 | 0.19583 | 0.18279 | 0.18726 |
| Ammonia % | 0.2510 | 0.3204 | 0.2177 | 0.2074 | 0.2014 |

| Metabolite | 12 | 13 | Min | Max | Std Dev | Average |
|---|---|---|---|---|---|---|
| Chloride M | 0.3522 | 0.2709 | 0.2683 | 0.4117 | 0.0428 | 0.3225 |
| Magnesium | 0.0041 | 0.0036 | 0.0031 | 0.0059 | 0.0009 | 0.0045 |
| M | | | | | | |
| Citrate M | 0.0019 | 0.0023 | 0.0005 | 0.0023 | 0.0006 | 0.0014 |
| Oxalate M | 0.00085 | 0.0014 | 0.0008 | 0.0021 | 0.0004 | 0.0013 |
| Sodium M | 0.1419 | 0.1645 | 0.1129 | 0.2604 | 0.0361 | 0.1520 |
| Phosphate M | 0.0554 | 0.0716 | 0.0430 | 0.0722 | 0.0084 | 0.0597 |
| Calcium M | 0.00148 | 0.00166 | 0.0009 | 0.0095 | 0.0024 | 0.0020 |
| Potassium % | 0.227659 | 0.20175 | 0.1596 | 0.2277 | 0.0184 | 0.1956 |
| Ammonia % | 0.2658 | 0.202 | 0.2014 | 0.3204 | 0.0317 | 0.2350 |

TABLE 3
Observed and Predicted RSS Values for Nutrients and Urine pH

| Group | 1 | 2 | 3 | 4 | 5 | 6 | 7 | 8 | 9 |
|---|---|---|---|---|---|---|---|---|---|
| RSS Ox Observed | 8.672 | 8.317 | 7.502 | 6.785 | 7.058 | 6.791 | 6.198 | 3.875 | 4.188 |
| RSS Mg Observed | 7.573 | 7.728 | 16.086 | 12.071 | 9.190 | 20.147 | 8.872 | 6.580 | 5.704 |

Uses Nutrients in Food Only

| | | | | | | | | | |
|---|---|---|---|---|---|---|---|---|---|
| Predicted RSS Ox | 8.677 | 8.677 | 6.879 | 6.910 | 7.194 | 5.791 | 4.894 | 3.985 | 4.554 |
| (LFF) | | | | | | | | | |
| Observed Urine pH | 6.183 | 6.223 | 6.354 | 6.375 | 6.209 | 6.668 | 6.302 | 6.177 | 6.288 |

| Group | 10 | 11 | 12 | 13 | Min | Max | Std Dev | Average |
|---|---|---|---|---|---|---|---|---|
| RSS Ox Observed | 4.137 | 4.316 | 4.183 | 6.213 | 3.875 | 8.672 | 1.698 | 5.865 |
| RSS Mg Observed | 6.267 | 7.616 | 7.606 | 11.643 | 5.704 | 20.147 | 4.231 | 9.485 |

Uses Nutrients in Food Only

| | | | | | | | | |
|---|---|---|---|---|---|---|---|---|
| Predicted RSS Ox (LFF) | 5.302 | 5.213 | 4.784 | 5.119 | | | | |
| Observed Urine pH | 6.253 | 6.352 | 6.424 | 6.708 | 6.177 | 6.708 | 0.169 | 6.335 |

TABLE 4
Observed and Predicted RSS Values for Metabolites,
Urine, and Urine pH

| | | | | | | | |
|---|---|---|---|---|---|---|---|
| Group | 1 | 2 | 3 | 4 | 5 | 6 | 7 |

Uses Metabolites in Urine and Urine pH

| | | | | | | | |
|---|---|---|---|---|---|---|---|
| Predicted RSS | 7.814 | 8.049 | 7.002 | 7.383 | 6.708 | 7.784 | 5.767 |
| Ox (LU) | | | | | | | |
| Predicted RSS | 7.776 | 9.616 | 12.818 | 11.743 | 9.728 | 16.299 | 8.885 |
| Mg (LUU) | | | | | | | |

Use Metabolites in Urine and Nutrients in Food

| | | | | | | | |
|---|---|---|---|---|---|---|---|
| Predicted RSS | 8.400 | 8.579 | 7.089 | 7.056 | 6.972 | 7.545 | 5.619 |
| Ox (LUF) | | | | | | | |

| | | | | | | |
|---|---|---|---|---|---|---|
| Group | 8 | 9 | 10 | 11 | 12 | 13 |

Uses Metabolites in Urine and Urine pH

| | | | | | | |
|---|---|---|---|---|---|---|
| Predicted RSS Ox (LU) | 4.707 | 4.277 | 4.836 | 4.632 | 4.122 | 5.299 |
| Predicted RSS Mg | 5.635 | 6.676 | 5.938 | 7.521 | 10.495 | 14.238 |
| (LUU) | | | | | | |

Use Metabolites in Urine and Nutrients in Food

| | | | | | | |
|---|---|---|---|---|---|---|
| Predicted RSS Ox | 3.795 | 4.230 | 4.871 | 4.699 | 4.145 | 5.315 |
| (LUF) | | | | | | |

The results from stepwise regression analysis show which nutrients, metabolites, and urine parameters are important for predicting oxalate and/or struvite stone formation. Utilizing the criteria of the EQUIL2 program, three models are developed for oxalate stone formation and one model for struvite stone formation. The results show that oxalate and/or struvite stone formation can be predicted from a small number of food nutrient, urine metabolite, and urine pH values compared to EQUIL2.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for predicting oxalate stone formation in an animal comprising:
determining the amount of selected nutrients in a food for consumption by the animal; and
predicting oxalate stone formation using a formula that equates the amount of the nutrients to the likelihood of oxalate stone formation.

2. The method of claim 1 wherein the nutrients are calcium, chloride, and phosphorous and the formula is (Formula 1):
LF=FA−(FB*potassium)+(FC*chloride)−(FD*calcium), where FA is from about
14 to about 26; FB is from about 9 to about 29; FC is from about 1 to about 5; and
FD is from about 1 to about 5, wherein the animal is likely to form oxalate stones if
LF is greater than or equal to 5.

3. The method of claim 1 wherein the animal is a companion animal.

4. The method of claim 1 wherein the animal is a canine.

5. The method of claim 1 wherein the animal is a feline.

6. A method for predicting oxalate stone formation in an animal comprising:
determining the amount of selected metabolites in a urine sample from the animal; and
predicting oxalate stone formation using a formula that equates the amount of the urine metabolites to the likelihood of oxalate stone formation.

7. The method of claim 6 wherein the metabolites are magnesium, citrate, and oxalate and the formula is (Formula 2):
LU=UA+(UB*magnesium)+(UC*oxalate)−(UD*citrate), where UA is from
about 0.6 to about 1.0; UB is from about 200 to about 600; UC is from about 2000 to
about 3600; and UD is from about 300 to about 600, wherein the animal is likely to
form oxalate stones if LU is greater than or equal to 5.

8. The method of claim 7 further comprising determining the amount of potassium in a food for consumption by the animal and the formula is (Formula 3):
LUF=UF+(UFA*magnesium)+(UFB*oxalate)−UFC*citrate)−
(UFD*potassium), where UF is from about 2 to about 9; UFA is from about 100 to
about 200; UFB is from about 1200 to about 3600; UFC is from about 200 to about
700; and UFD is from about 2 to about 6, wherein the animal is likely to form
oxalate stones if LUF is greater than or equal to 5.

9. The method of claim 6 wherein the animal is a companion animal.

10. The method of claim 6 wherein the animal is a canine.

11. The method of claim 6 wherein the animal is a feline.

12. A method for predicting struvite stone formation in an animal comprising:
determining the urine pH for the animal;
determining the amount of selected metabolites in a urine sample from the animal; and
predicting struvite stone formation using a formula that equates the urine pH and the amount of the urine metabolites to the likelihood of struvite stone formation.

13. The method of claim 12 wherein the metabolites are chloride, magnesium, and citrate and the formula is (Formula 4):
LUU=UU+(UUA*Urine pH)+(UUB*chloride)−UUC*magnesium)−
(UUD*citrate), where UU is from about −140 to about −180; UUA is from about 20
to about 30; UUB is from about 10 to about 30; UUC is from about 1100 to about
2900; and UUD is from about 1000 to about 2000, wherein the animal is likely to
form oxalate stones if LUU is greater than or equal to 10.

14. The method of claim 12 wherein the animal is a companion animal.

15. The method of claim 12 wherein the animal is a canine.

16. The method of claims 12 wherein the animal is a feline.

17. A device suitable for predicting oxalate and/or struvite stone formation in an animal comprising a means for predicting oxalate and/or struvite stone formation selected from the group consisting of one or more of Formula 1, Formula, 2, Formula 3, and Formula 4.

18. The device of claim 17 wherein the means accepts input from a user comprising, as appropriate for the Formula, a combination of at least 3 of potassium, chloride, calcium, magnesium, oxalate, citrate, and Urine pH and utilizes all or a subset of the input and one or more of Formula 1, Formula 2, Formula 3, and Formula 4 to predict oxalate and/or struvite stone formation in the animal.

19. The device of claim 17 wherein the means is a website, software program, calculator, or computer.

20. The device of claim 17 coupled to one or more analytical instruments capable of determining food nutrient content, urine metabolite concentration, or urine pH

21. A kit suitable for predicting oxalate and/or struvite stone formation in for an animal comprising in separate containers in a single package or in a virtual package, as appropriate for the kit component, a device of claim 17 and one or more of (1) a food suitable for animal consumption, (2) a urine pH diagnostic device, (3) a means for communicating information about or instructions for using urine pH diagnostic devices, and (4) a means for communicating information about or instructions for using the methods, devices, and kits of the present invention to predict oxalate and/or struvite stone formation in an animal.

22. A means for communicating information about or instructions for (1) using the methods, devices, and kits of the present invention to predict oxalate and/or struvite stone formation in an animal and (2) using urine pH diagnostic devices to predict urine pH comprising a document, digital storage media, optical storage media, audio presentation, or visual display containing the information or instructions.

23. The means for communicating of claim 22 selected from the group consisting of a displayed website, brochure, product label, package insert, advertisement, and visual display.

24. A method for predicting oxalate and struvite stone formation in an animal comprising a method of claim 1.

25. The method of claim 24 wherein the animal is a companion animal.

26. The method of claim 24 wherein the animal is a canine.

27. The method of claim 24 wherein the animal is a feline.
